(19) United States
(12) Patent Application Publication
(10) Pub. No.: US 2023/0210645 A1
(43) Pub. Date: Jul. 6, 2023

(54) Aerosol evacuation system with a barrier cover

(71) Applicants: Albert Jinli Chan, Cerritos, CA (US); Alex Chieh-Chang Chan, Cerritos, CA (US)

(72) Inventors: Albert Jinli Chan, Cerritos, CA (US); Alex Chieh-Chang Chan, Cerritos, CA (US)

(21) Appl. No.: 17/300,973

(22) Filed: Dec. 31, 2021

(51) Int. Cl. A61C 17/08; A61B 90/00

(52) U.S. Cl. CPC A61C 17/08; A61B 90/05

(57) ABSTRACT

The invention discloses an improved dental aerosol evacuation system equipped with a barrier cover subsystem in a dental procedure. Due to its dome-like special shape and other particular shapes, the barrier cover of the subsystem possesses a vacuum collecting capability that also physically blocks the spread of dispersed aerosols, blood components, and/or droplets during the dental procedures. At least a throughput connecting hole is connected from the curved barrier cover to the proximal end of the suction tubing of the subsystem. The vacuum collecting subsystem of the invention evacuates all or most of the collected unhealthy risk factors out of the treatment room completely for safety of patients, staff, and practitioners.

### FIELD OF THE INVENTION

The present invention is related to general uses of an improved aerosol evacuation system having a barrier cover subsystem in a dental procedure. The barrier cover subsystem of the invention has vacuum collecting capability by means of a conduit connection to a typical suction tubing possibly housed within the inner surface of an adjustable gooseneck tubing. The gooseneck tubing is in turn, possibly housed within a protective tubing. The disclosed vacuum collecting subsystem complements the typical dental aerosol evacuation procedures by evacuating most or all of the collected aerosols, droplets, and/or unwanted matter out of the treatment room via an internally connected vacuum suction tubing of the subsystem for the safety of patients, dental staff, and dentists.

### BACKGROUND OF THE INVENTION

Dental procedures may involve dental handpieces, ultrasonic scalers, 3-way air/water syringes, and/or one or more auxiliary supporting tools/appliances in a dental clinic. Dental procedures can generate aerosols and droplets which may contain saliva, blood, tiny particulates, blood components, unwanted matter, foreign material, and the like (collectively herein called ‘unhealthy risk factors’). Most of the unhealthy risk factors (including diseases) as listed collectively or individually can lead to the transmission of a variety of infectious diseases within the dental clinic facility; such infectious diseases may include COVID-19, tuberculosis, pneumonia, HIV, or the like. Among them, COVID-19 is caused by a coronavirus called SARS-2-CoV-2. Sometimes new variants emerge and disappear. Other times, new variants persist. One of the best ways for handling the COVID-19 virus situation is to prevent its transmission and/or to mitigate the diseases via medical devices or medical treatments. The current COVID-19 virus caused one of the world's deadliest pandemics and has killed over five million people worldwide.

To protect patients, dental staff, dentists, or other dental practitioners from any of these unhealthy risks factors, we developed, designed, and disclosed a dental subsystem particularly for the dental treatment room and is used along with the common dental facilities in a dental clinic. As disclosed in this invention, the barrier cover subsystem of the invention comprises of at least a barrier cover having solid interior cover surfaces for collecting and directing at least part or all of the unhealthy risk factors by means of sufficient suction forces derived directly by an attached/connected suction tubing of the disclosed subsystem, where the collected risk factors are mostly or completely suctioned through the suction (evacuating) tubing in the dental clinic that has a connected throughput hole conduit directly connected to the suction tubing, where the hole conduit is generally around the center portion of a barrier cover of the present subsystem.

The throughput hole conduit as defined herein is to straightly connect a throughput hole on the mentioned barrier cover to a proximal open section end of the suction tubing of the subsystem. In one embodiment, the throughput hole mechanism is securely inserted inside the proximal end space of the provided suction tubing via a heat shrink plastic cover from outside of the connection section. Hereby, the barrier cover subsystem becomes an essential part of the whole aerosol evacuation system in a dental procedure. The purpose of the barrier cover subsystem is for removing aerosols, droplets, blood, blood components, other unwanted matter, and the like (the so-called ‘unhealthy risk factors”) from the patient's mouth in the treatment room as appropriate and as fast as possible. Our system is unlike mobile dental suction units since their suctioned/filtered air may contain residual ‘unhealthy risk factors’ which can be exhausted back into the treatment room.

### SUMMARY OF THE INVENTION

It is one object of the present invention to provide a novel, unique dental device subsystem as a part of the dental procedures in a dental clinic, along with several dental procedures/processes that may involve dental handpieces, ultrasonic scalers, 3-way air/water syringes, and/or any auxiliary supporting tools/appliances in a dental clinic. This dental subsystem of the invention comprises a barrier cover with some solid cover surface having a small narrow throughput hole or conduit around the central portion of the cover. The narrow small throughput hole of the cover is internally connected to out-going evacuating suction tubing. The suction tubing can typically connect to a HVE (high volume evacuator) machine or apparatus for evacuation treatment. The cover physically blocks the spread of dispersed aerosols, droplets, and the like during the dental procedures in a dental facility in an effective and efficient manner. Thereafter the invented vacuum tubing subsystem possesses an open proximal end section of the tubing throughput securely and straightly connected to a hole conduit of the barrier cover. Its connecting setup of the invented subsystem is to suction all or most of the high risk factors or infective diseases (collectedly referred to aerosols, blood, droplets, tiny particulates, blood components, unwanted matter, and the like) from the interior side of a barrier cover surface out of the treatment room via the connected hole conduit mechanism as desired and as practicably needed.

In some embodiment of an improved aerosol evacuation dental system, it is provided a subsystem comprising of a barrier cover having a throughout hole, a suction tubing and an internally connecting conduit between the cover and the tubing for an activity of suctioning or vacuuming of unhealthy risk factors out of a treatment area. The suction tubing in this invention may be a PVC tubing or made of other comparable material.

Some aspects of the invention provide a subsystem that comprises at least one barrier cover having a throughout conduit hole, at least one suction tubing and at least an internal conduit connection between the at least one cover and the at least one tubing for an activity of suctioning or transporting/evacuating of unhealthy risk factors out of a treatment area more profoundly.

Here we disclose a method of physically blocking spread of dispersed aerosols and/or droplets from possibly spilling outside of a barrier cover during a dental procedure in a dental clinic. The subsystem comprises of at least one suction tubing and at least one curved barrier cover having solid interior inner surfaces, where the barrier cover has a throughput hole conduit around the center portion of the barrier cover, and wherein the hole conduit of the cover firmly attaches and connects to a proximal end section of the suction tubing, and wherein a proximal end section of the suction tubing is tightly wrapped around the hole conduit of the cover so the proximal end suction of the tubing is tightly pressurized inwards toward the throughput hole conduit to create a connected inner conduit between the barrier cover and the suction tubing.

Some aspects of the invention provide a method of using a subsystem of the invention to physically block the spread of saliva, blood, tiny particulates, blood components, unwanted matter, infectious diseases, COVID-19, variants, and the like in the dental treatment room.

In one aspect of the invention, the method herein possesses a connected inner conduit that is used to suction dispersed aerosols and/or droplets toward a high volume evacuator (HVE) apparatus via a suction tubing in order to clean and keep clean a dental treatment area.

Some aspects of the invention provide a connected inner conduit between the barrier cover and the suction tubing to swiftly and smoothly suction any dispersed aerosols and/or droplets from any interior solid surface of the cover barrier, wherein the connected inner conduit evacuates the unhealthy risk factors, aerosols, and/or droplets from the barrier cover toward the suctioning tubing. The inner conduit enables an effective and efficient suction power to pull unwanted aerosols, droplets, blood, saliva and the like adjacent to or within a treatment space in the dental clinic into the suction tubing via the connected inner conduit to dispose of any unwanted aerosols, droplets, saliva, blood, or the like outside of the treatment area. In one particular embodiment, the interior solid surface of the cover barrier has been treated sufficiently friction-free with respect to the unhealthy risk factors. This is to reduce the chance for or completely prevent the factors from sticking onto the interior surface during the suction procedures.

Some aspects of the invention provide a method that the throughput hole conduit of the barrier cover of the subsystem is snugly connected to an inner side of the proximal end section of the suction tubing of the subsystem for easy connection and/or disconnection.

Some aspects of the invention provide a distal end of the suction tubing being connected to an outlet of an existing HVE (high volume evacuator) which is commonly used in nearly every dental facility for the vacuum function feature or vacuum suction capability. Typically, the vacuum suction power of the HVE is reasonably or accurately adjustable by a dental practitioner according to his/her needs and requirements.

Some aspects of the invention provide the proximal end section of the suction tubing being expandable larger than the throughput hole conduit of the barrier cover to enable the expanded proximal tubing end section to tightly compress the hole conduit so the suction tubing is firmly attached to the hole conduit with intended suction capability.

Some aspects of the invention provide the barrier cover throughput hole of the subsystem being snugly connected to the proximal end section of the suction tubing of the subsystem for easy connection and disconnection, wherein the connecting function between the barrier cover and the suction tubing is similar to the connecting actions (plug-in or plug-out) of the suction tubing toward the HVE apparatus.

Some aspects of the invention provide a subsystem that further comprises of a gooseneck tubing, which optionally and conventionally allows the suction tubing to be flexibly manipulated and can be positioned by a practitioner directly over the patient's mouth/face.

Some aspects of the invention provide a subsystem further comprising of a gooseneck tubing which is commonly used to enclose or adjust the flexible suction tubing to be positioned appropriately adjacent to a desired place of a dental piece, an ultrasonic scalers, a 3-way air-water syringe, or other instrument.

Some aspects of the invention further provide the subsystem, wherein the gooseneck tubing is covered by a smooth tubing or tubing-like material that can be easily sterilized with disinfecting wipes.

Some aspects of the invention provide the subsystem, wherein the suction tubing of the subsystem invention can be sterilized by flushing the interior with a solution of liquid disinfection or something equally effective with the HVE system on.

Some aspects of the invention provide the subsystem, wherein the subsystem is supported by a clamp that is fixed in place to a dental chair or other fixture in the treatment room preferably with a solid equipment setup.

Some aspects of the invention provide the subsystem, wherein the subsystem is portable and/or movable, which can be supported firmly by a standalone round base support or other shape base support.

Some aspects of the invention provide the subsystem, wherein the aerosol evacuation device and the subsystem of a barrier cover along with a connected conduit is located adjacent and approximate a patient's treatment space in a dental clinic.

Some aspects of the invention provide the subsystem, wherein the inner surface or outer surface of the gooseneck tubing that surrounds the flexible suction tubing is sterilizable with a disinfecting wipe or solution.

Some aspects of the invention provide the subsystem, wherein the barrier cover is made of hard, transparent (mostly plastic) material and is disposable so as to reduce or mitigate a possibility of contamination and/or to eliminate the time needed for sterilization.

Some aspects of the invention provide the subsystem, wherein the barrier cover is made of hard, transparent material and can be autoclaved or cold sterilized for reuse. The hard, transparent material can be made of plastic material or non-plastic material.

Some aspects of the invention provide the subsystem, wherein the barrier cover is slightly elongated shape, round shaped, or otherwise suitably shaped so that the barrier cover covers most of the face/mouth treatment area with a purpose of physically blocking the spread of unhealthy risk factors during the dental procedures. For example, the barrier cover can be made by molding or air blowing. Another example is to cut a transparent ball or elongated hard balloon shaped cover at almost through the center zone. Then the barrier cover is provided a throughput hole at approximately the center of the cover to be snugly connected to the proximal end section of the suction tubing to form the subsystem of the invention for the HVE (high volume evacuator) in the treatment room.

### BRIEF DESCRIPTION OF THE DRAWINGS

Additional objects and features of the present invention will become more apparent and the disclosure itself will be best understood from the following Detailed Description of the Exemplary Embodiments, when read with reference to the accompanying drawings.

FIG. 1 shows an improved aerosol evacuation dental system having a barrier cover subsystem in an improved dental procedure. A clamp is used to fix the subsystem to a dental chair or other fixture.

FIG. 2 shows a standalone supporting post and round base that supports the improved aerosol evacuation subsystem of the invention. The weighted base allows the subsystem of the invention to be portable and/or movable from one area or dental operatory to another.

FIG. 3 shows a cross-sectional view of at least two concentric tubings of an improved aerosol evacuation dental systems of the invention.

FIG. 4 shows a connecting mechanism description of the disclosed subsystem for creating a straight throughput conduit between the barrier cover portion and the suction tubing portion of the invented aerosol evacuation subsystem.

FIG. 5 shows another suitably shaped configuration or pattern of the barrier cover to fit a person's face, which is non dome-shaped (more like oval shape).

### DETAILED DESCRIPTION OF THE EXEMPLARY EMBODIMENTS

The preferred embodiments of the present invention described below relate particularly to a preparation of an improved dental aerosol evacuation subsystem having two basic components and an internally connecting mechanism in between. The connecting mechanism is joined or turned “on” when the subsystem is in treatment operation by connecting and turning on the suction tubing to the HVE apparatus. On the other hand, after each dental operation, the suction tubing part of the subsystem, could be disconnected, turned off, or un-plugged from the HVE apparatus. In one example, the barrier cover has a vacuum collecting capability along the internal surface 19 of a curved barrier cover 3 that has a solid internal surface 19 that physically blocks the spread of dispersed aerosols and/or droplets from spilling/flying toward the outside of the barrier cover 3 during the dental procedures, and the collection of dispersed aerosols and/or droplets, etc. from the internal cover side 19 of the barrier cover 3 is thereafter disposed out of the treatment area via a working suction tubing 1 of the vacuum collecting subsystem 13. The purpose of this subsystem is to evacuate all or most of the collected aerosols, droplets and/or unwanted matter out of the treatment room from the barrier cover 3 via a connected throughput vacuum suction tubing part of the subsystem for the safety of patients, staff, and dentists when the suction tubing 1 is connected to a HVE and turned on. In one embodiment, the internal cover surface 19 of the barrier cover 3 has a smooth surface to facilitate inward flow of the unhealthy risk factors toward the suction tubing.

Furthermore, after the connecting conduit mechanism between the suction tubing and the HVE port is off after each treatment operation, the connection of the suction tubing is temporarily stopped. The barrier cover 3 can be separated from the suction tubing 1 and disposed thereafter.

A disease, such as the infectious disease COVID-19, coronavirus, or a similar disease, is a particular abnormal condition that negatively affects the structure or function of all or part of an organism, and that is not due to any immediate external injury. It is generally categorized as an unhealthy risk factor in this disclosure.

A dental clinic is usually divided into multiple rooms (at least two); one of which is the “treatment room” which is referred to where a patient would be treated under an aerosol evacuation device by a practitioner. Another room or extra space of the office is where equipment or apparatus/instruments are maintained; the spare room can also be used as storage spaces. The main HVE equipment is located nearby for the convenience of a practitioner for the dental treatment. The practitioner can easily connect and plug-in the suction tubing of the subsystem into the HVE suction source/port during dental treatments; the practitioner can also easily disconnect or unplug the suction tubing of the subsystem out of the HVE port when the job is completed.

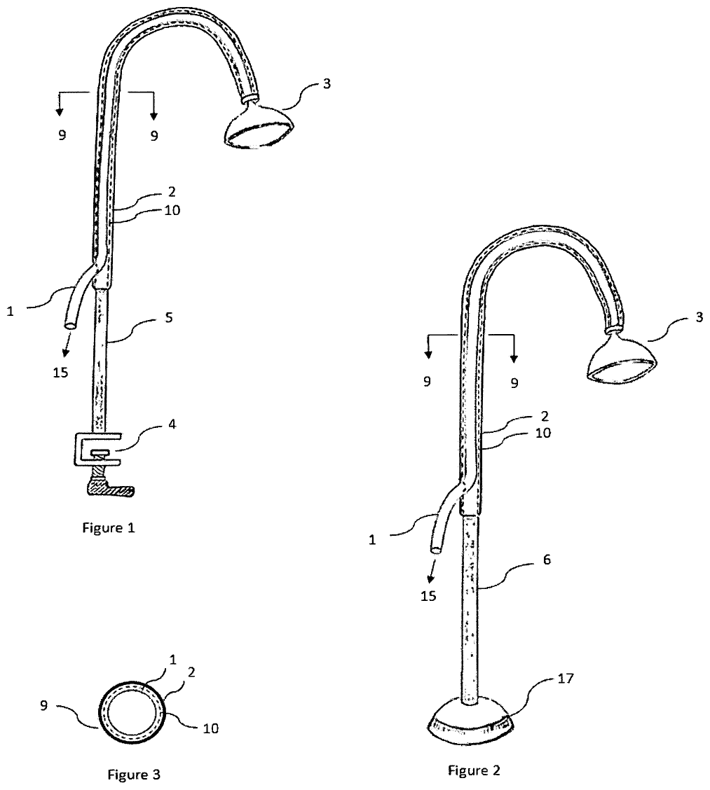

The “barrier” or a ‘barrier cover’ as used in this invention may include any solid dome-shaped, solid semi-spherical shaped, solid elongated shaped, or other suitably shaped cover that conveniently and appropriately covers or approximates a patient's face/mouth area for dental treatment to mitigate or minimize the spread of infectious diseases or other health risk factors. The barrier cover 3 as shown in FIG. 1 has a continuous rim 14 along its cover edge so none or few foreign material may accidently enter or leave the empty cover volume 20, where the internal cover volume 20 is an open space so the practitioner can conduct his/her treatment jobs using one or more of the instruments, such as dental handpieces, ultrasonic scalers, 3-way air/water syringes, and/or any auxiliary supporting tools/appliances. The subsystem of the invention includes particularly two parts (i.e., barrier cover 3 and the suction tubing 1) either connected during the dental operation, or not connected when not in dental operation. The first part of the subsystem hereby is a barrier cover 3 which can be dome-like shaped. The second part of the subsystem is a suction tubing 1 which possesses suction and evacuation power when connected to or plugged into a HVE (high volume evacuation) system. In some embodiment, the barrier cover 3 of the present invention has a solid and relatively smooth interior surface that allows for relatively smooth airflow that carries and removes the health risk factors out of the barrier cover via a connected vacuum tubing 1 through the HVE apparatus.

FIG. 1 shows an improved aerosol evacuation dental system having a barrier cover subsystem 13 in a dental procedure, wherein the barrier cover subsystem of the invention is on a fixable supporting post 5 that supports the subsystem 13 of the barrier cover connecting to an out-going suction tubing. In one embodiment, the clamp 4 is equipped on the bottom part of the subsystem to fix the subsystem in place for use. In one embodiment, the clamp 4 is used to fix the subsystem to a dental chair or other fixture in the dental treatment room. A gooseneck type tubing is also used to adjust the appropriate location of the suction tube 1.

As in this FIG. 1, the suction tubing 1 is inside the gooseneck tube 10. This allows the suction subsystem to be flexible and can be positioned by the practitioner directly over the patient's mouth and/or face or another aerosol producing source. The joint area of the suction tubing and the gooseneck tubing is sealed so that no leaking of aerosols and droplets, which may contain saliva, blood, tiny particulates, blood components, unwanted matter, and the like (collectively herein called ‘unhealthy risk factors’), is possible. By the same token, the suction tubing is the main tubing for providing suction force from the distal end outside of the treatment area.

In one particular embodiment, the gooseneck tubing is covered by a smooth tubing 2 that can be easily sterilized with disinfecting wipes. In a similar manner, the suction tubing 1 can be sterilized by flushing the interior with solution of liquid disinfection with the HVE system on.

FIG. 2 shows a standalone supporting base 17 with a sturdy stem 6 to supports the improved aerosol evacuation subsystem of the invention. In another embodiment, the mobile subsystem is easily and conveniently relocated by a practitioner for use. In another embodiment, the base 17 can be a round shape or other appropriate shape, which is a heavily weighted base to fully support the subsystem, the base support post, and other auxiliary parts associated with the invention.

FIG. 1 and FIG. 2 shows a truncated (a portion of) suction tubing 1, where the distal end of the tubing directly follows the direction 15 toward the HVE plug-in port for providing suction force. It is the plug-out action from the HVE port that discontinues the suction force for the suction tubing.

FIG. 3 shows one example of at least two concentric tubings of an improved aerosol evacuation dental systems in a cross-sectional view 9-9 of FIG. 1, where two or more tubings (suction tubing 1, smooth tubing 2, or gooseneck tubing 10) are layered almost concentrically inside along the tubing central lines longitudinally. It is similar to the layering of an undersea electric cables system. In one embodiment, the inner suction tubing 1 lies longitudinally alongside the gooseneck tubing 10, which lies longitudinally within a smooth thin tubing 2. The suction tubing 1 can be sterilized by flushing the interior with a solution of liquid disinfectant or something of similar effect with the HVE system on.

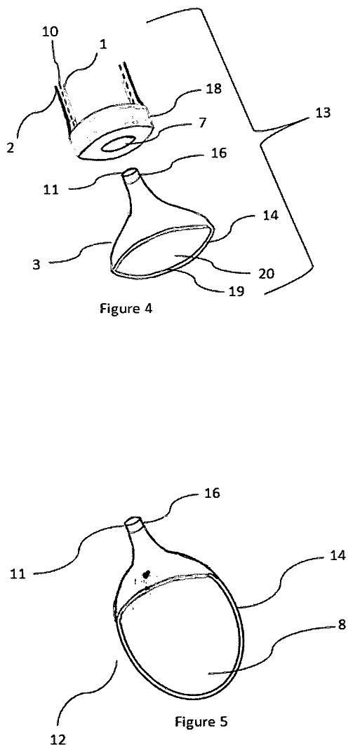

FIG. 4 shows a connecting mechanism for creating a throughput conduit between the barrier cover 3 and the suction tubing subsystem 13 of the invention. In FIG. 4, the barrier cover 3 shows a lamp-shaped (or other non dome-shaped) configuration, which has a continuous rim 14 on the cover 3 and an open space 20, where most of the treatment operations would be conducted. The distal end 11 of the barrier cover 3 has a narrowed down portion 16 but still has an open throughput space 11. This allows one to lightly squeeze the distal end 11 into the proximal end section 7 of the suction tubing 1.

In some embodiment, the peripheral space 7 close to the peripheral end of the suction tubing 1 may be slightly larger than the outer distal end surface 18 of the cover. Therefore, the outer distal surface 18 of the distal end 11 of the barrier cover 3 can still be relatively easy to insert into the inner space of the proximal end section of the suction tubing 1. In one particular embodiment, a silicone, rubber, or plastic ring cover and/or end cap is to be placed over the inner throughput connection section to create an air-leak-proof and/or water-leak-proof connection. This is to ensure adequate suction of the unhealthy risk factors from the barrier cover toward the HVE side upon working conditions (the direction of arrow 15). In another embodiment, the dome connector 16 is firmly squeezed into the end of the proximal section of the suction tubing 1 for intended treatment operations.

Our system commonly utilizes an adjustable gooseneck feature that surrounds the suction tubing to stabilize the tubing position. In one embodiment as shown in FIG. 3 and FIG. 4, the suction tubing 1 is located inside the gooseneck tubing 10 concentrically). The distal end of the suction tubing 1 has a connecting mechanism that is supposed to quickly connect to (or plug-in) a HVE suction source/port (which is not shown), or disconnect (or unplug) from the HVE when needed (not shown either).

In another embodiment, the gooseneck feature can wrap around the suction tubing, since this type of gooseneck tubing is supported and strengthened by some steel wires or so forth. This allows the suction system or suction head to be flexibly adjusted, relocated, and/or positioned by the practitioner as appropriate over the patient's mouth/face or other treatment area. The gooseneck can be covered by smooth thin tubing that can be easily sterilized after use with disinfecting wipes or the like. The suction tubing 1 can be sterilized by flushing the interior with a solution of liquid disinfection with the HVE system on.

The barrier cover system is positioned and held near a dental patient's face and/or mouth during treatment or near another aerosol generating source, where the subsystem combines a barrier cover (i.e., the hard cover surface to physically block the spread of health risk factors) for the patient and the barrier cover with a throughput hole mechanism that is firmly attached to a proximal section of the vacuum tubing via, for example, a gooseneck tubing support, other inserted semi-flexible support, flexible wire, a silicone, rubber, or plastic ring cover and/or end cap in order to place the barrier cover subsystem 13 (including the barrier cover and the attached suctioning tubing) in place securely that approximates the treatment area during the dental operations.

FIG. 5 shows another suitably shaped pattern of the barrier cover 12 to fit a person's face, which is non dome-shaped having void space 8 and barrier cover rim 14. This special barrier cover 12 may better fit the face or cheek area of a special patient for better collecting the unhealthy risk factors.

The barrier cover is generally built of any solid and transparent plastic material, such as acrylic or other similar materials. Its construction or manufacturing method is not limited by any special configuration, method, or material. It is generally built to be impermeable to any gas, gel, or liquid penetration and resist fogging or becoming blurry. As referred to in this invention, any cover with plastic-like internal cover surface 19 to suction and vacuum any unhealthy risk factors away from the treatment area may provide unforeseen health benefits to patients.

Our system utilizes and connects to an existing HVE (high volume evacuator) which is usually used in nearly every dental facility for the vacuum function. The current invention with a barrier cover on a patient allows aerosols and droplets to be vacuumed and evacuated of the treatment room. The barrier cover is essentially dome or dome-like shaped to fully cover the patients' mouth and/or face as much as possible.

To protect patients, dental staff, dentists, or other dental practitioners from any of the previously mentioned unhealthy risks factors, we developed, designed, and disclosed a dental subsystem for the dental treatment room and is used along with the common dental facilities in a dental clinic. As disclosed in this invention, the barrier cover subsystem of the invention comprises at least a solid-surface barrier cover for collecting at least the majority of the unhealthy risk factors by applying certain sufficient suction forces derived directly from the attached suction tubing of the subsystem, where the collected unhealthy risk factors is mostly or completely suctioned through the suction (evacuating) tubing having a connected throughput hole mechanism located at around the center portion of the barrier covers. The throughput hole mechanism is to openly connect the mentioned barrier cover hole to a proximal open end section of the suction tubing of the barrier cover subsystem.

In one embodiment, the throughput hole mechanism is securely inserted inside the proximal end of the suction tubing via a silicone, rubber, or plastic ring cover and/or end cap from the outside of the connection section. The barrier cover system is held near or very close to a dental patient during treatment or near another aerosol generating source, where the subsystem combines a barrier cover (i.e., the hard cover surface to physically block the spread of health risks) for the patient and the barrier cover with a throughput hole mechanism that is firmly attached to a proximal section of the vacuum tubing via, for example, a gooseneck tubing support, other inserted semi-flexible support, or the silicone, rubber, or plastic ring cover and/or end cap, including heat-shrinkable ring cover, in order to place the barrier cover subsystem (including the barrier cover and the attached suctioning tubing) in place securely that approximates the treatment area during the dental operations. Hereby, the barrier cover subsystem becomes an essential part of the whole aerosol evacuation system in a dental procedure, particularly during the COVID-19 pandemic. The purpose of the barrier cover subsystem is for removing aerosols, droplets, blood, blood components, and the like or other unwanted matters (the so-called ‘unhealthy risk factors”) from the patient's mouth and/or other aerosol generating source in the dental clinic as appropriately, as completely, as conveniently, and as quickly as possible.

The barrier cover of the invention is transparent or is very much transparent, and does not resist or hinder the dental practitioner's working view. The barrier cover is also developed and designed to be disposable. In some embodiments, the barrier of the invention designed to be reusable after autoclaving or cold sterilization. The barrier is roughly dome shaped or dome-like shaped to cover the patient's mouth and/or face. In some embodiments, the shape of the barrier of the invention could be specially designed to fit and cover the individual shape of the patient's mouth and face. Its primary purpose is to offer a face-mouth cover with vacuum capability to physically block and also evacuate the spread of any infectious disease, disposed aerosols and droplets during the dental procedures.

Although the present invention has been described with reference to specific details of certain embodiments thereof, it is not intended that such details should be regarded as limitations upon the scope of the invention except as and to the extent that they are included in the accompanying claims. Many modifications and variations are possible in light of the above disclosure.

What is claimed is:

1. A method of physically blocking spread of dispersed aerosols and/or droplets from possibly spilling toward outside of a barrier cover during a dental procedure in a dental aerosol evacuation device system, the system comprising of a subsystem including at least one suction tubing and at least one curved barrier cover having solid inner surfaces, where the barrier cover has a throughput hole conduit near center portion of the barrier cover, and wherein the hole conduit of the cover is firmly attached and connected to a proximal end section of the suction tubing, and wherein a proximal part of the suction tubing is tightly wrapped around said hole conduit so the proximal end suction tubing is tightly pressurized inwards toward said throughput hole conduit to create a connected inner conduit between the barrier cover and the suction tubing.

2. The method of claim 1, where the method of the subsystem further physically blocks the spread of saliva, blood, tiny particulates, blood components, unwanted matter, infectious diseases, COVID-19, variants, and the like.

3. The method of claim 1, wherein the connected inner conduit is used to suction dispersed aerosols and/or droplets toward a high vacuum evacuator (HVE) apparatus and out of the treatment room in order to clean and keep clean a dental treatment area.

4. The method of claim 1, wherein the connected inner conduit between the barrier cover and the suction tubing is used to swiftly and smoothly suction any dispersed aerosols and/or droplets from any interior solid surface of the barrier cover, wherein the connected inner conduit evacuates the unhealthy aerosols and/or droplets from the barrier cover toward the suctioning tubing, wherein said inner conduit enables an effective and efficient suction power to pull unwanted aerosols, droplets, blood, saliva and the like adjacent to or within a treatment space in a dental clinic into the suction tubing via the connected inner conduit to dispose of any unwanted aerosols, droplets, saliva, blood, or the like outside of the treatment area.

5. The method of claim 1, wherein the throughput hole conduit of the barrier cover of the subsystem is snugly connected to an inner side of the proximal end of the suction tubing of the subsystem for easy connection and disconnection.

6. The method of claim 1, wherein a distal end of the suction tubing is connected to an outlet of an existing HVE (high volume evacuator) which is commonly used in nearly every dental facility for a vacuum function feature or vacuum suction capability, wherein a vacuum suction power of the HVE is reasonably or accurately adjustable by a dental practitioner according to his/her needs and requirements.

7. The method of claim 1, wherein the proximal part of the suction tubing may be expandable to larger than the throughput hole conduit of the barrier cover, and wherein a silicone, rubber, or plastic ring cover and/or end cap is attached to the proximal end of the suction tubing, to enable the expanded proximal tubing section to tightly compress the hole conduit so the suction tubing is firmly attached to said hole conduit.

8. The method of claim 1, wherein the barrier cover throughput hole of the subsystem is snugly connected to the proximal end of the suction tubing of the subsystem for easy connection and disconnection, wherein the connecting function between the barrier cover and the suction tubing is similar to connecting actions (plug-in or plug-out) of the suction tubing with respect to the FIVE apparatus operations.

9. The method of claim 1, wherein the subsystem may further comprise a gooseneck tubing, which allows the suction tubing to be flexibly manipulated and can be positioned by a practitioner directly over a patient's mouth/face for better dental treatment and for optimal removal of unhealthy risk factors.

10. The method of claim 1, wherein the subsystem further comprises of a gooseneck tubing which is commonly used to enclose or adjust the flexible suction tubing to position the subsystem appropriately adjacent to the patient's mouth and/or face or other aerosol generating source.

11. The method of claim 1, wherein the gooseneck tubing is covered by a smooth tubing that can be easily sterilized with disinfecting wipes.

12. The method of claim 1, wherein the suction tubing can be sterilized by flushing the interior with a solution of liquid disinfectant with the HVE system on.

13. The method of claim 1, wherein the subsystem is supported by a clamp that is fixed in place to a dental chair or other fixture in the treatment room possibly with a solid equipment setup.

14. The method of claim 1, wherein the subsystem is movable and is supported by a standalone heavy base support.

15. The method of claim 1, wherein the aerosol evacuation device and the subsystem of a barrier cover along with a connected conduit is located adjacent to and approximate a patient's treatment space in the dental clinic.

16. The method of claim 1, wherein an inside surface or outer surface of the gooseneck tubing that surround the flexible suction tubing is sterilizable with a disinfecting wipe or solution.

17. The dental aerosol evacuation device of claim 1, wherein the barrier cover is made of hard transparent plastic material and is disposable so as to reduce a possibility of contamination and to eliminate time needed for sterilization.

18. The dental aerosol evacuation device of claim 1, wherein the barrier cover is made of hard transparent material that can be autoclaved or cold sterilized for reuse.

19. The dental aerosol evacuation device of claim 1, wherein a shape of the barrier cover is slightly elongated shape, round shaped, or otherwise suitably shaped so that the barrier cover covers most of the face/mouth treatment area with a purpose of physically blocking the spread of unhealthy risk factors during the dental procedures.

20. The dental aerosol evacuation device of claim 1, wherein the subsystem comprises at least one barrier cover having a throughout conduit hole, at least one suction tubing and at least an internal conduit connection between the at least one barrier cover and the at least one tubing for an activity of suctioning or transporting/evacuating of unhealthy risk factors out of a treatment area more profoundly.
